(19) United States
(12) Patent Application Publication
(10) Pub. No.: US 2020/0282096 A1
(43) Pub. Date: Sep. 10, 2020

(54) DEVICES, SYSTEMS AND METHODS FOR THE ELECTROCHEMICAL MODULATION OF ODORANT MOLECULES

(71) Applicant: Duke University, Durham, NC (US)

(72) Inventors: Edgard NGABOYAMAHINA, Durham, NC (US); Claire DE MARCH, Durham, NC (US); Jeffrey GLASS, Durham, NC (US); Brian STONER, Durham, NC (US); Hiroaki MATSUNAMI, Durham, NC (US)

(21) Appl. No.: 16/764,146

(22) PCT Filed: Nov. 13, 2018
(86) PCT No.: PCT/US2018/060782

§ 371 Date: May 14, 2020

Related U.S. Application Data
(60) Provisional application No. 62/585,622, filed on Nov. 14, 2017.

(51) Int. Cl. A61L 9/16; C25B 3/00

(52) U.S. Cl. CPC A61L 9/16; C25B 3/00

(57) ABSTRACT

The present invention provides devices, systems and methods for electrochemically modulating functional groups associated with a specific odorant molecule for purposes of altering the smell associated with the specific odorant molecule.

### CROSS REFERENCE TO RELATED APPLICATION

This application claims priority to and the benefit of U.S. Provisional Application No. 62/585,622, filed Nov. 14, 2017, which is hereby incorporated by reference in its entirety.

### FIELD OF THE INVENTION

The present invention provides devices, systems and methods for electrochemically modulating functional groups associated with a specific odorant molecule for purposes of altering the smell associated with the specific odorant molecule.

### BACKGROUND

Odor management is an important challenge across different activities including increasing the presence of a desired odorant, and reducing the presence of an undesired odorant (e.g., managing odorant pollution from sanitation facilities, industry, restaurant or agriculture). Notably, olfactory nuisance in unimproved sanitation facilities is one of the primary reasons for open defecation for up to one-billion people worldwide.

Common approaches to odor management rely on either hiding the bad smell through exposure to a pleasant odorant or absorbing some of the malodorant compounds on active carbon or lipocalin proteins. The success of such techniques is limited by their non-universality (they are not applicable to all malodorous molecules) and the need for maintenance.

Improved techniques for odor management are needed.

The present invention addresses this need.

### SUMMARY

Experiments conducted during the course of developing embodiments for the present invention determined that specific functional groups associated with specific odorant molecules can be modulated (e.g., selective reduction and/or oxidation) through electrochemical modulation, thereby changing the smell associated with the odorant molecule. Indeed, such experiments demonstrated successful modulation of an odorant molecule having a carboxylic acid functional group (e.g., butyric acid) to butanol through electrochemical modulation. Such experiments demonstrated successful modulation of p-cresol odorant molecules through electrochemical modulation.

Accordingly, the present invention provides devices, systems and methods for electrochemically modulating functional groups associated with a specific odorant molecule for purposes of altering the smell associated with the specific odorant molecule.

In certain embodiments, the present invention provides devices comprising one or more electrochemically active surface areas, wherein each of the one or more electrochemically active surface areas is configured to electrochemically modulate the smell associated with a specific odorant molecule.

In some embodiments, the devices have therein an air in-flow portion configured to direct air having odorant molecules to the one or more electrochemically active surface areas. In some embodiments, the devices have therein an air out-flow portion configured to direct electrochemically modulated odorant molecules out of the devices.

In some embodiments, the devices can be configured such that each can be programmed to electrochemically modulate the smell associated with specific odorant molecules so as to obtain a desired smell within a setting.

In some embodiments, the devices are configured such that each can be directed to electrochemically modulate the smell associated with specific odorant molecules so as to inhibit an undesired smell within a setting.

Such devices are not limited to a particular type or kind of electrochemically active surface area. In some embodiments, the one or more of the electrochemically active surface areas is a carbon-based electrochemically active surface area. In some embodiments, the carbon-based electrochemically active surface area is a graphite-based electrochemically active surface area. In some embodiments, the one or more of the electrochemically active surface areas comprises an electrode. In some embodiments, the one or more of the electrochemically active surface areas is a gas diffusion electrode. In some embodiments, the one or more of the electrochemically active surface areas is any type of electric cell capable of modulating the chemical structure of odorant molecules upon contact with such odorant molecules. In some embodiments, the one or more of the electrochemically active surface areas is a voltaic cell. In some embodiments, the one or more of the electrochemically active surface areas is in contact with an acidic solution. In some embodiments, the one or more of the electrochemically active surface areas is in contact with sulfuric acid. In some embodiments, the one or more of the electrochemically active surface areas is in contact with a basic/alkaline substance.

In some embodiments, the one or more of the electrochemically active surface areas are configured to apply an electric current to contacted odorant molecules which electrochemically modulates one or more functional groups associated with the one or more odorant molecules.

In some embodiments, the one or more of the electrochemically active surface areas are configured to electrochemically modulate ester functional groups to one or more of aldehyde, ketone, alkane, carboxylic acid, and alcohol derivatives.

In some embodiments, the one or more of the electrochemically active surface areas are configured to electrochemically modulate linear terpene functional groups to terpene derivatives.

In some embodiments, the one or more of the electrochemically active surface areas are configured to electrochemically modulate cyclic terpene functional groups to terpene derivatives.

In some embodiments, the one or more of the electrochemically active surface areas are configured to electrochemically modulate aromatic functional groups to aromatic derivatives.

In some embodiments, the one or more of the electrochemically active surface areas are configured to electrochemically modulate amine functional groups amide derivatives.

In some embodiments, the one or more of the electrochemically active surface areas are configured to electrochemically modulate carboxylic acid functional groups to one or more of aldehyde, ketone, alkane, ester, and alcohol derivatives.

In some embodiments, the one or more of the electrochemically active surface areas are configured to electrochemically modulate alcohol functional groups to one or more of aldehyde, carboxylic acid, alkane, ester, and ketone derivatives.

In some embodiments, the one or more of the electrochemically active surface areas are configured to electrochemically modulate aldehyde functional groups to one or more of aldehyde, carboxylic acid, alkane, ester, and ketone derivatives.

In some embodiments, the one or more of the electrochemically active surface areas are configured to electrochemically modulate thiol functional groups to sulfide derivatives.

In some embodiments, the one or more of the electrochemically active surface areas are configured to electrochemically modulate ketone functional groups to one or more of aldehyde, carboxylic acid, alkane, ketone, and ester derivatives.

In some embodiments, the one or more of the electrochemically active surface areas are configured to electrochemically modulate lactone functional groups to one or more of diol derivatives.

In some embodiments, the one or more electrochemically active surface areas are configured to apply a constant electric potential (e.g., at approximately −0.6 V) to contacted odorant molecules.

In some embodiments, the one or more electrochemically active surface areas are configured to apply cyclic voltammetry (e.g., from approximately −0.2 V to +1.1 V) to contacted odorant molecules.

In some embodiments, the one or more electrochemically active surface area modulates the chemical structure of odorant molecules through reducing functional groups on the one or more odorant molecules.

In some embodiments, the one or more electrochemically active surface area modulates the chemical structure of odorant molecules through oxidizing functional groups on the one or more odorant molecules.

In certain embodiments, present invention provides methods for modulating one or more types of odorant molecules within a setting, comprising providing a device as described herein, and directing one or more types of odorant molecules within the setting with the one or more electrochemically active surface areas of the device.

In some embodiments, the modulation of the one or more types of odorant molecules results in a modulated smell within the setting.

In some embodiments, the one or more of the odorant molecules are selected from geranyl acetate, methyl formate, methyl acetate, methyl proprionate, methyl propanoate, fructone, methyl butyrate, methyl butanoate, ethyl acetate, hexyl acetate, ethyl methylphenylglycidate, ethyl butyrate, ethyl butanoate, isoamyl acetate, pentyl butyrate, pentyl butanoate, pentyl pentanoate, octyl acetate, benzyl acetate, and methyl anthranilate. In some embodiments, the one or more of the one or more electrochemically active surfaces are configured to electrochemically modulate ester functional groups associated with the one or more odorant molecules.

In some embodiments, the one or more of the odorant molecules are selected from myrcene, geraniol, nerol, citral, lemonal, geranial, neral, citronellal, citronellol, linalool, and nerolidol. In some embodiments, the one or more of the one or more electrochemically active surfaces are configured to electrochemically modulate linear terpene functional groups associated with the one or more odorant molecules.

In some embodiments, the one or more of the odorant molecules are selected from limonene, camphor, menthol, carvone, terpineol, alpha-lonone, thujone, and eucalyptol. In some embodiments, the one or more of the one or more electrochemically active surfaces are configured to electrochemically modulate cyclic terpene functional groups associated with the one or more odorant molecules.

In some embodiments, the one or more of the odorant molecules are selected from benzaldehyde, eugenol, cinnamaldehyde, ethyl maltol, vanillin, anisole, anethole, estragole, and thymol. In some embodiments, the one or more of the one or more electrochemically active surfaces are configured to electrochemically modulate aromatic functional groups associated with the one or more odorant molecules.

In some embodiments, the one or more of the odorant molecules are selected from trimethylamine, putrescine, diaminobutane, cadaverine, pyridine, indole, and skatole. In some embodiments, the one or more of the one or more electrochemically active surfaces are configured to electrochemically modulate amine functional groups associated with the one or more odorant molecules.

In some embodiments, the one or more of the odorant molecules is butyric acid. In some embodiments, the one or more of the one or more electrochemically active surfaces are configured to electrochemically modulate carboxylic acid functional groups associated with the one or more odorant molecules.

In some embodiments, the one or more of the odorant molecules are selected from p-cresol, furaneol, 1 1-hexanol, cis-3-hexen-1-ol, and menthol. In some embodiments, the one or more of the one or more electrochemically active surfaces are configured to electrochemically modulate alcohol functional groups associated with the one or more odorant molecules.

In some embodiments, the one or more of the odorant molecules are selected from acetaldehyde, hexanal, cis-3-hexenal, furfural, hexyl cinnamaldehyde, isovaleraldehyde, anisic aldehyde, and cuminaldehyde. In some embodiments, the one or more of the one or more electrochemically active surfaces are configured to electrochemically modulate aldehyde functional groups associated with the one or more odorant molecules.

In some embodiments, the one or more of the odorant molecules are selected from thioacetone, allyl thiol, benzyl mercaptan, (methylthio)methanethiol, ethanethiol, ethyl-mercaptan, 2-methyl-2-propanethiol, butane-1-thiol, grapefruit mercaptan, methanethiol, and furan-2-ylmethanethiol. In some embodiments, the one or more of the one or more electrochemically active surfaces are configured to electrochemically modulate thiol functional groups associated with the one or more odorant molecules.

In some embodiments, the one or more of the odorant molecules are selected from cyclopentadecanone, dihydrojasmone, 6-acetyl-2,3,4,5-tetrahydropyridine, oct-1-en-3-one, and 2-acetyl-1-pyrroline. In some embodiments, the one or more of the one or more electrochemically active surfaces are configured to electrochemically modulate ketone functional groups associated with the one or more odorant molecules.

In some embodiments, the one or more of the odorant molecules are selected from gamma-nonalactone, gamma-decalactone, delta-octalactone, jasmine lactone, massoia lactone, wine lactone, and sotolon. In some embodiments, the one or more of the one or more electrochemically active surfaces are configured to electrochemically modulate lactone functional groups associated with the one or more odorant molecules.

In some embodiments, the one or more of the odorant molecules are selected from dimethyl trisulfide, zinc phosphide, methylphosphine, dimethylphosphine, diacetyl, acetoin, nerolin, tetrahydrothiophene, 2,4,6-trichloroanisole, and odorant molecules having a substituted pyrazine functional group.

Such methods are not limited to a particular setting. In some embodiments, the setting is an industrial setting. In some embodiments, the the setting is within a room no larger than 250 square feet.

In certain embodiments, the present invention provides systems or kits comprising one or more of the devices described in claim 1 and instructions for use of the one or more devices.

### BRIEF DESCRIPTION OF THE DRAWINGS

FIG. 1: Electrochemical characterization of graphite electrode in sulfuric acid with 10% butyric acid. Fourier-transform infrared (FTIR) spectroscopy confirmed the generation of butanol (circled peak) following treatment of butyric acid by cyclic voltammetry. Results indicate that a cathodic vertex lower than −0.4V vs Ag/AgCl is required to drive the electrochemical reduction of butyric acid to butanol.

FIG. 2 shows the reaction equation for generating 4-hydroxybenzaldehyde by p-cresol oxidation. In the present example, molecular oxygen and catalytic functional groups are anodically produced at the surface of a graphite electrode.

FIG. 3 shows cyclic voltammetry of H₂SO₄+0.1% p-cresol. p-cresol oxidation peak was identified at 0.55 V with cyclic voltammetry for 0.1% p-cresol in 0.5N H₂SO₄. Oxygen saturation was shown to have a catalytic effect as indicated by the increased oxidation peak.

FIG. 4 shows a proposed mechanism for p-cresol modulation. Treatment by successive oxidation and reduction steps can lead to a mixture of several compounds.

FIG. 5 shows the odor modulation pathway, from the air intake to the exhaust.

### DETAILED DESCRIPTION

Several studies have linked human perception of odorant molecules to the odorant molecules physical structure and its specific receptors (see, Khan et al. (2007) Journal of Neuroscience 27(37):10015; Kermen et al. (2011) Sci Rep 1:206; Keller et al., (2007) Nature 449(7161):468-472; Menashe et al. (2007) PLoS Biol 5(11):e284).

Odors are complex mixtures of chemical species, and so contain many constituent odorant molecules. The biological olfactory system is a remarkable sensor having many olfactory cells or odorant receptors, but not very many different types of olfactory cells. The characterization of a scent or odor is typically through the combined response of many of the receptors.

There are a large number of odorant molecules having different polarization characteristics and molecular weights. Hitherto, a variety of methods have been developed for modulating odorant molecules (e.g., modulating the smell associated with a specific odorant molecule). Generally, the methods for modulating odorant molecules are broadly classified into a biological method, a chemical method, a physical method, or a sensory method. Among odorant molecules, short-chain fatty acids and amines, having high polarity, can be reduced through a chemical method; i.e., neutralization. Sulfur-containing compounds such as thiol can be reduced through a physical method; i.e., adsorption.

Known means for modulating the smell associated with a particular odorant molecule include the following: a composition containing a porous substance, an aminopolycarboxylic acid, and a metal (see, JP2002-153545); a silk burned product supporting a catalyst such as platinum (see, WO 2005/007287); a deodorant containing, as an active ingredient, allyl heptanoate, ethyl vanillin, methyl dihydrojasmonate, raspberry ketone, or eugenol (see, JP2005-296169); and use of an aromatic component such as amylcinnamaldehyde, ethyl cinnamate, 2-cyclohexylpropanal (Pollenal II), geranyl acetone, cis-3-hexenyl heptanoate, cis-3-hexenyl hexanoate, 3-methyl-3-butenyl 2,2-dimethylpropionate (Lomilat), methylheptenone, valencene, dimethyltetrahydrobenzaldehyde (Triplal or Ligustral), cis-jasmon, acetylcedrene, benzyl acetate, geraniol, orange recovery flavor, or a plant extract of Dipterocarpaceae (see, JP2005-296169; JP2008-136841).

According to the aforementioned means, an odorant molecule is modulated by decreasing the amount of target odorant molecule through adsorption/decomposition or by means of an aromatic. However, the combination of adsorption and decomposition of such an odorant molecule is not immediately effective, since the decrease of the amount thereof requires a long period of time. Use of an aromatic also has drawbacks in that the odor of the aromatic itself sometimes causes an unpleasant sensation to users, and the aromatic tends to mask odor of substances other than the target odorant molecule. Indeed, the success for such techniques in reducing or modulating odorant molecules is not optimal.

The electrochemical reactivity of odorant molecules has yet to be extensively investigated. As noted, most common odorant molecules have a chemical structure consisting of, for example, carbon chains ending with functional groups (e.g., ester, linear terpene, cyclic terpene, aromatic, amine, carboxylic acid, alcohol, aldehyde, thiol, ketone, and lactone) that render the odorant molecule with a particular odor (e.g., smell, fragrance, etc). Indeed, such functional groups signify the odor character of a given odorant molecule. As such, a modulation or change of the chemical functional group on a common carbon backbone can result in dramatically different smell perceptions, from waxy to fruity for instance (see, de March C A, et al., (2015) Flavour and Fragrance Journal, 30: 331-410).

Experiments conducted during the course of developing embodiments for the present invention determined that specific functional groups associated with specific odorant molecules can be modulated (e.g., selective reduction and/or oxidation) through electrochemical modulation, thereby changing the smell associated with the odorant molecule. Indeed, such experiments demonstrated successful modulation of an odorant molecule having a carboxylic acid functional group (e.g., butyric acid) to butanol through electrochemical modulation. Such experiments demonstrated successful modulation of p-cresol odorant molecules to 4-hydroxybenzaldehyde through electrochemical modulation.

Accordingly, the present invention provides devices, systems and methods for electrochemically modulating functional groups associated with a specific odorant molecule for purposes of altering the smell associated with the specific odorant molecule.

Indeed, in certain embodiments, the present invention provides methods for electrochemically modulating the chemical structure of odorant molecules through contacting one or more odorant molecules with an electrochemically active surface area.

Such embodiments are not limited to a particular type or kind of electrochemically active surface area. In some embodiments, the electrochemically active surface area is a carbon-based electrochemically active surface area. In some embodiments, the electrochemically active surface area is graphite-based electrochemically active surface area. In some embodiments, the electrochemically active surface area is an electric cell. In some embodiments, the electrochemically active surface area is an electrode. In some embodiments, the electrochemically active surface area is a gas diffusion electrode. In some embodiments, the electrochemically active surface area is any type of electric cell capable of modulating the chemical structure of odorant molecules upon contact with such odorant molecules. In some embodiments, the electrochemically active surface area is a voltaic cell. In some embodiments, the electrochemically active surface area is in contact with an acidic substance. In some embodiments, the electrochemically active surface area is in contact with sulfuric acid (e.g., H₂SO₄; HCl). In some embodiments, the electrochemically active surface area is in contact with a basic/alkaline substance.

Such electrochemically active surface areas are not limited to a particular manner of electrochemically modulating the chemical structure of odorant molecules. In some embodiments, the electrochemically active surface area is configured to apply an electric current to contacted odorant molecules. In some embodiments, the electrochemically active surface area is configured to apply a constant electric potential (e.g., at approximately −0.6 V) to contacted odorant molecules. In some embodiments, the electrochemically active surface area is configured to apply cyclic voltammetry (e.g., from approximately −0.2 V to +1.1 V) to contacted odorant molecules.

Such electrochemically active surface areas are not limited to a particular manner of contacting odorant molecules for purposes of electrochemically modulating the chemical structure of the contacted odorant molecules. In some embodiments, odorant molecules passively contact the electrochemically active surface areas (e.g., through ambient air contact). In some embodiments, odorant molecules are actively directed to contact the electrochemically active surface areas (e.g., through an airflow system configured to direct such odorant molecules to the electrochemically active surface area) (e.g., through a ventilation system configured to direct such odorant molecules to the electrochemically active surface area).

In some embodiments, the electrochemically active surface area modulates the chemical structure of odorant molecules through reducing functional groups on the odorant molecules. In some embodiments, the electrochemically active surface area modulates the chemical structure of odorant molecules through oxidizing functional groups on the odorant molecules.

Such embodiments are not limited to electrochemically modulating specific functional groups associated with specific odorant molecules.

In some embodiments, the electrochemically active surface area is configured to electrochemically modulate ester functional groups on specific odorant molecules. Such embodiments are not limited to a specific manner of electrochemically modulating ester functional groups on specific odorant molecules. In some embodiments, ester functional groups on specific odorant molecules are electrochemically modulated to one or more of aldehyde, ketone, alkane, carboxylic acid, and alcohol derivatives. Examples of odorant molecules having an ester functional group that signifies the odor character of the given odorant molecule include, but are not limited to, geranyl acetate, methyl formate, methyl acetate, methyl proprionate, methyl propanoate, fructone, methyl butyrate, methyl butanoate, ethyl acetate, hexyl acetate, ethyl methylphenylglycidate, ethyl butyrate, ethyl butanoate, isoamyl acetate, pentyl butyrate, pentyl butanoate, pentyl pentanoate, octyl acetate, benzyl acetate, and methyl anthranilate.

In some embodiments, the electrochemically active surface area is configured to electrochemically modulate linear terpene functional groups on specific odorant molecules. Such embodiments are not limited to a specific manner of electrochemically modulating linear terpene functional groups on specific odorant molecules. In some embodiments, linear terpene functional groups on specific odorant molecules are electrochemically modulated to terpene derivatives (see, Larsen, et al., 1998 Chemosphere, Vol. 37, No. 6, pages 1207-1220). Examples of odorant molecules having a linear terpene functional group that signifies the odor character of the given odorant molecule include, but are not limited to, myrcene, geraniol, nerol, citral, lemonal, geranial, neral, citronellal, citronellol, linalool, and nerolidol.

In some embodiments, the electrochemically active surface area is configured to electrochemically modulate cyclic terpene functional groups on specific odorant molecules. Such embodiments are not limited to a specific manner of electrochemically modulating cyclic terpene functional groups on specific odorant molecules. In some embodiments, cyclic terpene functional groups on specific odorant molecules are electrochemically modulated to terpene derivatives (see, Larsen, et al., 1998 Chemosphere, Vol. 37, No. 6, pages 1207-1220). Examples of odorant molecules having a cyclic terpene functional group that signifies the odor character of the given odorant molecule include, but are not limited to, limonene, camphor, menthol, carvone, terpineol, alpha-lonone, thujone, and eucalyptol.

In some embodiments, the electrochemically active surface area is configured to electrochemically modulate aromatic functional groups on specific odorant molecules. Such embodiments are not limited to a specific manner of electrochemically modulating aromatic functional groups on specific odorant molecules. In some embodiments, aromatic functional groups on specific odorant molecules are electrochemically modulated to aromatic derivatives. Examples of odorant molecules having an aromatic functional group that signifies the odor character of the given odorant molecule include, but are not limited to, benzaldehyde, eugenol, cinnamaldehyde, ethyl maltol, vanillin, anisole, anethole, estragole, and thymol.

In some embodiments, the electrochemically active surface area is configured to electrochemically modulate amine functional groups on specific odorant molecules. Such embodiments are not limited to a specific manner of electrochemically modulating amine functional groups on specific odorant molecules. In some embodiments, amine functional groups on specific odorant molecules are electrochemically modulated to amide derivatives. Examples of odorant molecules having an amine functional group that signifies the odor character of the given odorant molecule include, but are not limited to, trimethylamine, putrescine, diaminobutane, cadaverine, pyridine, indole, and skatole.

In some embodiments, the electrochemically active surface area is configured to electrochemically modulate carboxylic acid functional groups on specific odorant molecules. Such embodiments are not limited to a specific manner of electrochemically modulating carboxylic acid functional groups on specific odorant molecules. In some embodiments, carboxylic acid functional groups on specific odorant molecules are electrochemically modulated to one or more of aldehyde, ketone, alkane, ester, and alcohol derivatives. Examples of odorant molecules having a carboxylic acid functional group that signifies the odor character of the given odorant molecule include, but are not limited to, butyric acid.

In some embodiments, the electrochemically active surface area is configured to electrochemically modulate alcohol functional groups on specific odorant molecules. Such embodiments are not limited to a specific manner of electrochemically modulating alcohol functional groups on specific odorant molecules. In some embodiments, alcohol functional groups on specific odorant molecules are electrochemically modulated to one or more of aldehyde, carboxylic acid, alkane, ester, and ketone derivatives. Examples of odorant molecules having an alcohol functional group that signifies the odor character of the given odorant molecule include, but are not limited to, p-cresol, furaneol, 1 1-hexanol, cis-3-hexen-1-ol, and menthol.

In some embodiments, the electrochemically active surface area is configured to electrochemically modulate aldehyde functional groups on specific odorant molecules. Such embodiments are not limited to a specific manner of electrochemically modulating aldehyde functional groups on specific odorant molecules. In some embodiments, aldehyde functional groups on specific odorant molecules are electrochemically modulated to one or more of aldehyde, ketone, alkane, ester, and carboxylic acid derivatives. Examples of odorant molecules having an aldehyde functional group that signifies the odor character of the given odorant molecule include, but are not limited to, acetaldehyde, hexanal, cis-3-hexenal, furfural, hexyl cinnamaldehyde, isovaleraldehyde, anisic aldehyde, and cuminaldehyde.

In some embodiments, the electrochemically active surface area is configured to electrochemically modulate thiol functional groups on specific odorant molecules. Such embodiments are not limited to a specific manner of electrochemically modulating thiol functional groups on specific odorant molecules. In some embodiments, thiol functional groups on specific odorant molecules are electrochemically modulated to sulfide derivatives. Examples of odorant molecules having a thiol functional group that signifies the odor character of the given odorant molecule include, but are not limited to, thioacetone, allyl thiol, benzyl mercaptan, (methylthio)methanethiol, ethanethiol, ethyl-mercaptan, 2-methyl-2-propanethiol, butane-1-thiol, grapefruit mercaptan, methanethiol, and furan-2-ylmethanethiol.

In some embodiments, the electrochemically active surface area is configured to electrochemically modulate ketone functional groups on specific odorant molecules. Such embodiments are not limited to a specific manner of electrochemically modulating ketone functional groups on specific odorant molecules. In some embodiments, ketone functional groups on specific odorant molecules are electrochemically modulated to one or more of aldehyde, carboxylic acid, alkane, ketone, and ester derivatives. Examples of odorant molecules having a ketone functional group that signifies the odor character of the given odorant molecule include, but are not limited to, cyclopentadecanone, dihydrojasmone, 6-acetyl-2,3,4,5-tetrahydropyridine, oct-1-en-3-one, and 2-acetyl-1-pyrroline.

In some embodiments, the electrochemically active surface area is configured to electrochemically modulate lactone functional groups on specific odorant molecules. Such embodiments are not limited to a specific manner of electrochemically modulating lactone functional groups on specific odorant molecules. In some embodiments, lactone functional groups on specific odorant molecules are electrochemically modulated to diol derivatives. Examples of odorant molecules having a lactone functional group that signifies the odor character of the given odorant molecule include, but are not limited to, gamma-nonalactone, gamma-decalactone, delta-octalactone, jasmine lactone, massoia lactone, wine lactone, and sotolon.

Examples of odorant molecules having a functional group that signifies the odor character of the given odorant molecule include, but are not limited to, dimethyl trisulfide, zinc phosphide, methylphosphine, dimethylphosphine, diacetyl, acetoin, nerolin, tetrahydrothiophene, 2,4,6-trichloroanisole, and odorant molecules having a substituted pyrazine functional group.

In some embodiments, the electrochemically active surface area configured to electrochemically modulate only one type of functional group on an odorant molecule. In some embodiments, the electrochemically active surface area configured to electrochemically modulate multiple types of functional group on various types of odorant molecules. In some embodiments, the electrochemically active surface area can be reconfigured to enhance, inhibit, provide, diminish any type of functional group on an odorant molecule (e.g., a user can re-configure the electrochemically active surface area depending on the desired smell or lack of smell for a particular setting).

In some embodiments, the electrochemically active surface area is configured to electrochemically modulate one or more types of functional groups on one or more types of odorant molecules with the purpose of modulating the aroma within a setting. For example, in some embodiments, the electrochemically active surface area is configured to electrochemically modulate one or more types of functional groups within odorant molecules associated with unpleasant smell (thereby removing and/or diminishing the presence of the unpleasant smell in a setting). For example, in some embodiments, the electrochemically active surface area is configured to electrochemically modulate one or more types of functional groups within odorant molecules associated with pleasant smell (thereby enhancing, removing and/or diminishing the presence of the pleasant smell in a setting). For example, in some embodiments, the electrochemically active surface area is configured to electrochemically modulate one or more types of functional groups within odorant molecules for purposes of effectuating a desired aroma within a setting.

Such electrochemically active surface areas are not limited to a particular size. In some embodiments, the size of the electrochemically active surface area is such that it is able to accomplish any desired amount of odorant molecule chemical structure modulation. For example, in some embodiments, the size of the electrochemically active surface area is such that it is able to accomplish a desired amount of odorant molecule chemical structure modulation within any setting (e.g., small room, large room, house setting, warehouse setting, etc.) (e.g., larger than 250 square feet) (e.g., larger than 1000 square feet) (e.g., less than 250 square feet).

In certain embodiments, the present invention provides devices for electrochemically modulating functional groups associated with a specific odorant molecule for purposes of altering the smell associated with the specific odorant molecule. Such devices are not limited to a particular structure or design. In some embodiments, the devices have therein an electrochemically active surface area (as described herein). In some embodiments, the device further comprises an air in-flow portion configured to direct air having odorant molecules to the electrochemically active surface area. In some embodiments, the device further comprises an air out-flow portion configured to direct electrochemically modulated odorant molecules out of the device. In some embodiments, the devices are closed (e.g., odorant molecules are actively imported into the closed setting, electrochemically modulated through contact with the electrochemically active surface area, and actively exported out of the closed setting). In some embodiments, the devices are open (e.g., odorant molecules are electrochemically modulated through passive contact with the electrochemically active surface area).

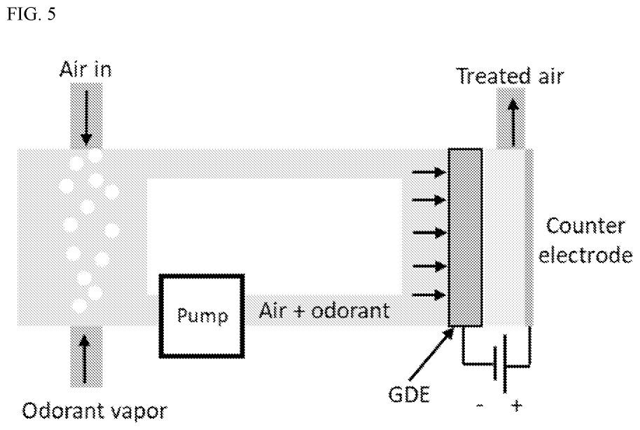

FIG. 5 shows an embodiment of a device of the present invention. As can be seen, the device has therein an intake region for passively receiving air and odorant molecules and a pump for directing such received air molecules to a gas diffusion electrode serving as the electrochemically active surface area. As shown, the treated air (e.g., air comprising electrochemically modulated odorant molecules) is exported from the device to the environment.

The devices of the present are not limited to a particular size. In some embodiments, the size of the device is such that it is able to accomplish any desired amount of odorant molecule electrochemical modulation within any setting (e.g., small room, large room, house, warehouse, building, etc.).

In some embodiments, the devices are configured for battery operation. In some embodiments, the devices are configured for connection with an alternating current (AC) power supply for operation. In some embodiments, the devices are configured for perpetual operation. In some embodiments, the devices are configured for use as needed. In some embodiments, the devices can be programmed to be operated upon occurrence of an event (e.g., the use of a bathroom) (e.g., upon detection of movement in a setting).

In some embodiments, the devices have more than one electrochemically active surface area, and a user can selectively operate more than one electrochemically active surface area depending on a smell or lack of smell for a particular setting.

In certain embodiments, the present invention provides systems having one or more devices for electrochemically modulating functional groups associated with a specific odorant molecule for purposes of altering the smell associated with the specific odorant molecule and instructions for utilizing such devices.

In certain embodiments, the present invention provides systems having one or more electrochemically active surface areas as described herein and instructions for utilizing such electrochemically active surface areas. In some embodiments, the systems further comprise an airflow system for directing air comprising odorant molecules to the one or more electrochemically active surface areas.

The systems, methods and devices of the present invention are not limited to particular uses.

In some embodiments, the systems, methods and devices are used to alter the smell within a particular setting. In some, the systems, methods and devices can be used to reduce and/or inhibit and/or prevent an unpleasant smell within a particular setting (e.g., a sanitation setting). For example, the systems, methods and devices can be implemented in a bathroom to reduce, inhibit and/or prevent unpleasant smells associated with bathrooms. For example, the systems, methods and devices can be implemented in an industrial setting to reduce, inhibit and/or prevent unpleasant smells associated with industrial settings.

In some embodiments, the systems, methods and devices are used to provide any desired smell or plurality of smells to a particular setting. In such embodiments, the systems, methods and devices are configured to generated the desired smell through electrochemical modulation of specific odorant molecules within an ambient setting.

In some embodiments, the systems, methods and devices can operate in unison with particular forms of media (e.g., radio, television, theatrical, etc.) to enhance such forms of media with desired smells. For example, the systems, methods and devices can be used to provide a user of such media with a smell associated with the media (e.g., a television could operate in unison with the systems, methods and devices to provide the smell of a particular situation occurring in a television program (e.g., a floral smell could be provided as a flower shop is shown in a television program) (e.g., a fecal smell could be provided as a unsanitary bathroom is shown in a television program).

### EXAMPLES

### Example I

This example demonstrates methods for calculating redox potentials for odorant molecules.

Calculating redox potentials is equal to calculating free energies of the reaction, according to the relationship

ΔG=−nFE, (1)

where ΔG is the Gibbs free energy for a reaction, E is the redox potential for the reaction, n is the number of electrons transferred in the reaction, and F is the Faraday constant. Since free energy is state function, a thermodynamic cycle is generally applied to obtain the reaction free energy in solution (see, Marenich, A. V.; et al., Physical Chemistry Chemical Physics 2014, 16 (29), 15068-15106), as shown in Table 1.

TABLE 1
Thermodynamic cycle for calculating Gibbs free
energy change of a reduction reaction in solution
by calculating that in gas state and the solvation
free energies of species.
$\begin{matrix}\begin{matrix}{\sum }_{i} {R}_{i} ( g ) + n {e}^{-} ( g ) {}^{Δ G ( g )} {\sum }_{j} {P}_{j } ( g ) \\ {\sum }_{i} Δ {G}_{s} ( {R}_{i} ) ↓ ↓ Δ {G}_{s} ( {e}^{-} ) = 0 ↓ {\sum }_{j} Δ {G}_{s} ( {P}_{j} )\end{matrix} \\ {\sum }_{i} {R}_{i} ( s ) + n {e}^{-} ( s ) {}^{Δ G ( s )} {\sum }_{j} {P}_{j} ( s )\end{matrix}$

R and P refer to reactants and products, respectively. ΔG is the reaction free energy and ΔGₛ is the solvation free energy.

The reaction free energy in solution is thus expressed as

ΔG(s)=ΔG(g)+ΣⱼΔGₛ(Pⱼ)−ΣᵢΔGₛ(Rᵢ), (2)

where ΔG(s) is the reaction free energy in solution, ΔG(g) is the reaction free energy in gas state, ΔGₛ(Pⱼ) is the solvation free energy for product Pⱼ, and ΔGₛ(Rᵢ) is the solvation free energy for reactant Rᵢ.

To obtain the gas-state reaction free energy ΔG^{o}(g) in standard state, the free energies of all compounds involved in the reaction are calculated, except that the free energy of electron in standard state is 0 kcal/mol from the ion convention with Boltzmann statistics (see, Bartmess, J. E., The Journal of Physical Chemistry 1994, 98 (25), 6420-6424). Such experiments applied hybrid functional Becke (see, Becke, A. D., The Journal of chemical physics 1993, 98 (7), 5648-5652), 3-parameter, Lee-Yang-Parr (see, Lee, C.; et al., Physical Review B 1988, 37 (2), 785-789) (B3LYP) with 6-31+G(d,p) basis set (see, Hehre, W. J.; et al., The Journal of Chemical Physics 1972, 56 (5), 2257-2261; Clark, T.; et al., Journal of Computational Chemistry 1983, 4 (3), 294-301; Frisch, M. J.; et al., The Journal of Chemical Physics 1984, 80 (7), 3265-3269) to optimize the geometry of each compound in G09 software (see, Frisch, M. J.; et al., Gaussian 09, Revision C.01, Gaussian, Inc., Wallingford Conn., 2010). The frequency is also calculated at this level to acquire the thermal correction to Gibbs free energy (see, Roth, H. G.; et al., Synlett 2016, 27 (05), 714-723). Next the single point energy is calculated at the level of coupled-cluster singles and doubles plus perturbative triples (see, Raghavachari, K.; et al., Chemical Physics Letters 1989, 157 (6), 479-483) (CCSD(T)) with the basis of aug-cc-pVTZ (see, Dunning Jr., T. H., The Journal of Chemical Physics 1989, 90 (2), 1007-1023). The final gas-state Gibbs free energy is the sum of single point energy from CCSD(T)/aug-cc-pVTZ and the thermal correction to Gibbs free energy from B3LYP/6-31+G(d,p) (see, Silva, P. J.; et al., Computational and Theoretical Chemistry 2011, 966 (1), 120-126; Isegawa, M.; et al., Journal of Chemical Theory and Computation 2016, 12 (5), 2272-2284). For the solvation free energy ΔGₛ, it is generally calculated with implicit solvent model or explicit solvent model (see, Skyner, R. E.; et al., Physical Chemistry Chemical Physics 2015, 17 (9), 6174-6191). However, the experimental solvation free energy is used here because it is readily available for these molecules (see, Shivakumar, D.; et al., Journal of Chemical Theory and Computation 2010, 6 (5), 1509-1519). Note that a Gibbs free energy correction (1.89 kcal/mol) should be added to change the substance from 1 atm (standard state for gas) to 1 mol/L (standard state for solution) (see, Arumugam, K.; et al., Minerals 2014, 4 (2), 345-387). The reaction free energy in solution ΔG^{o}(s) is thus obtained from Eq 2, where the superscript means standard state.

With ΔG^{o}(s), the absolute standard redox potential ΔE_{abs}⁰ for the reaction is calculated from Eq 1. Furthermore, the standard redox potential relative to silver chloride electrode (Ag/AgCl) reference electrode ΔE_{rel,Ag/AgCl}⁰ is represented as

ΔE_{rel,Ag/AgCl}^{o}=ΔE_{abs}^{o}−ΔE_{abs}^{o}(SHE)−ΔE_{rel,SHE}^{o}(Ag/AgCl), (3)

where ΔE_{abs}^{o}(SHE) is the absolute standard electrode potential of standard hydrogen electrode (SHE) and ΔE_{rel,SHE}^{o}(Ag/AgCl) is the standard potential of Ag/AgCl electrode with respect to SHE. To obtain the redox potential relative to Ag/AgCl electrode ΔE_{rel,Ag/AgCl} at the experimental condition, the Nernst equation is applied for the dilute solution as

$$Δ {E}_{re 1 , Ag \text{/} AgCl} = Δ {E}_{re 1 , Ag \text{/} AgCl}^{o} - \frac{RT}{nF} ln \frac{{Π}_{j} [ {P}_{j} ]}{{Π}_{i} [ {R}_{i} ]} , \qquad ( 4 )$$

where R is the gas constant, T is the temperature, and [Pⱼ] and [Rᵢ] are the concentrations of product j and product i, respectively.

In such experiments, three redox reactions were investigated, which are the reduction reaction from butyric acid to butanol (Reaction 1), the reduction reaction from butyric acid to butanal (Reaction 2) and the oxidation reaction from p-cresol to 4-hydroxybenzaldehyde (Reaction 3). The mechanisms for these three reactions are las follows:

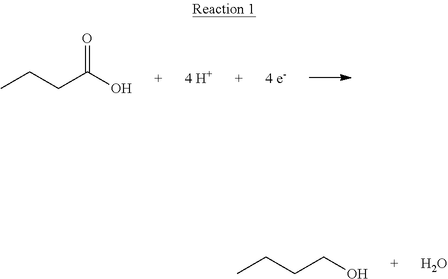

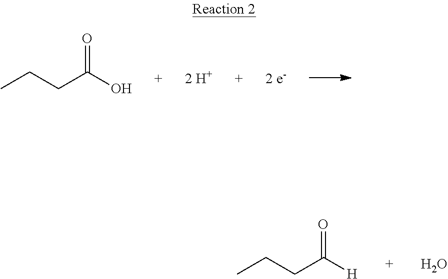

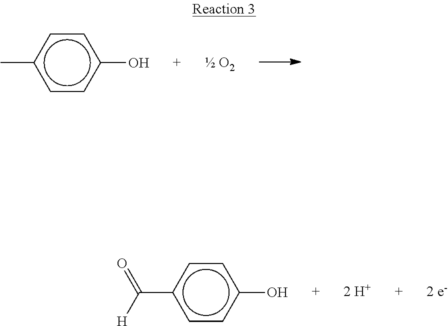

The gas-state Gibbs free energy and the solvation free energy for each substance involved in three reactions, which are applied to calculate reaction free energy in solution with Eq 2, are shown in Table 2. The ΔE_{abs}^{o}(SHE) and ΔE_{rel,SHE}^{o}(Ag/AgCl) are 4.28 V and 0.197 V, respectively (see, Bard, A. J.; et al., Standard potentials in aqueous solution. M. Dekker: New York, 1985; Kelly, C. P.; et al., The Journal of Physical Chemistry B 2006, 110 (32), 16066-16081). In the experimental condition, the concentrations of butyric acid, butanol, butanal, p-cresol, 4-hydroxybenzaldehyde are all 0.001 mol/L and PH is 0.5 for three reactions. The ΔE_{rel,Ag/AgCl} and ΔE_{rel,Ag/AgCl} for all reactions are thus easily calculated according to Eqs 3 and 4. As shown in Table 3, the reduction potentials at experimental condition for Reaction 1 and Reaction 2 are −0.19 V and −0.34 V, respectively, and the oxidation potential at experimental condition for Reaction 3 is 1.17 V.

TABLE 2
Gas-state Gibbs Free Energies (G(g)) and Experimental
Solvation Free Energies (ΔGₛ), in Units of kcal/mol,
of All Substances Involved in Three Reactions.

| Substances | G(g) (kcal/mol) | ΔGₛ (kcal/mol) |
|---|---|---|
| butyric acid | −192751.67 | −6.36 |
| butanol | −146327.36 | −4.72 |
| butanal | −145582.15 | −3.18 |
| p-cresol | −217184.46 | −6.14 |
| 4-hydroxybenzaldehyde | −263583.17 | −10.48 |
| water | −47903.19 | −6.30 |
| proton | −6.28 | −265.90 |
| oxygen gas | −94194.64 | \ |

TABLE 3
Redox Potentials (in Units of V) Relative to Ag/AgCl Reference
Electrode at the Standard State (ΔE_{rel,Ag/AgCl}^{o}) and at the
Experimental Condition (ΔE_{rel,Ag/AgCl}) for Three Reactions.

| Potential (V) | Reaction 1 | Reaction 2 | Reaction 3 |
|---|---|---|---|
| ΔE_{rel,Ag/AgCl}^{o} | −0.16 | −0.32 | 1.14 |
| ΔE_{rel,Ag/AgCl} | −0.19 | −0.34 | 1.17 |

### Example II

This example demonstrates the successful modulation of an odorant molecule having a carboxylic acid functional group (e.g., butyric acid) to butanol through electrochemical modulation.

Among other malodorous compounds, butyric acid has been identified as a strong contributor to human fecal odor (see, Zaleski, A.; et al., Przeglad Gastroenterologiczny 2013, 8 (6), 350-353; Chappuis Charles, J. F.; et al., Flavour and Fragrance Journal 2015, 31 (1), 95-100).

Previous experiments demonstrated that electrogenerated surface functional groups on boron-doped diamond can be stabilized by cyclic voltammetry (CV) allowing continuous catalytic reduction reactions.

Experiments were next conducted that expanded this finding to graphite (different type of carbonaceous materials with similar sp2 C on its surface) to study the electrochemical reduction of butyric acid. Changes in voltammetric behavior were observed and assigned to the formation surface functional groups by activation, but also to the increase in real surface area, likely due to roughening. Electrode reactions are always heterogeneous processes and their productivity is directly proportional to the electrochemically active surface area.

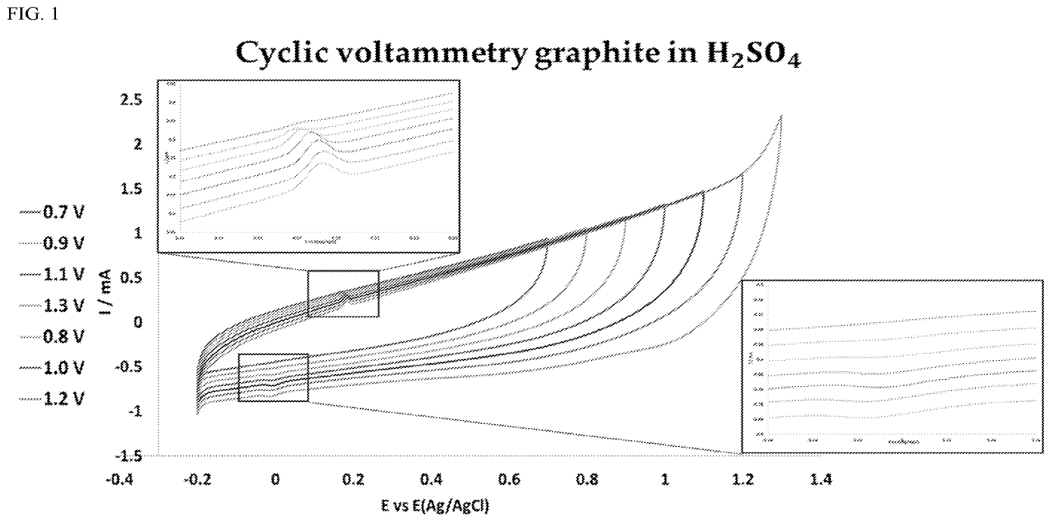

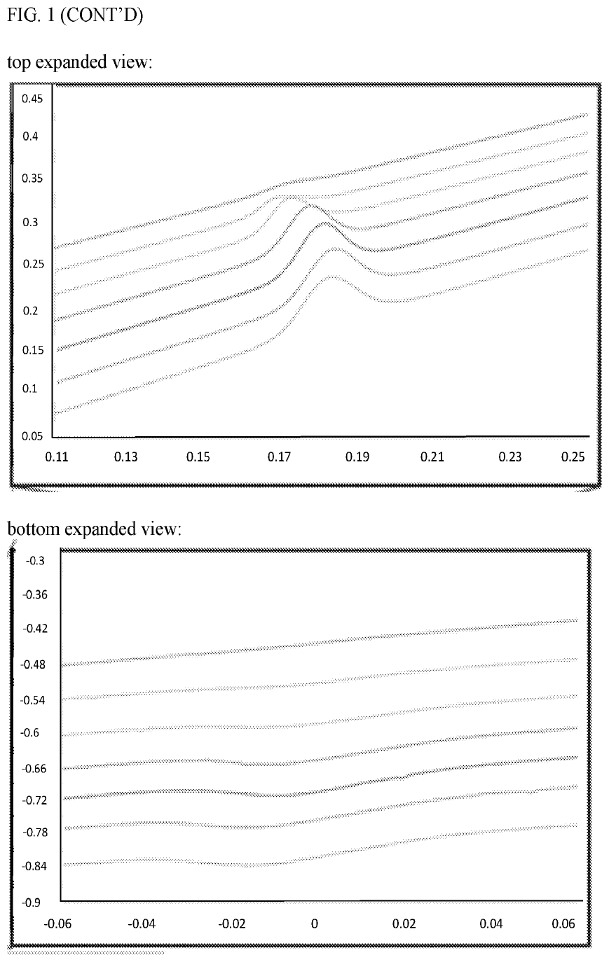

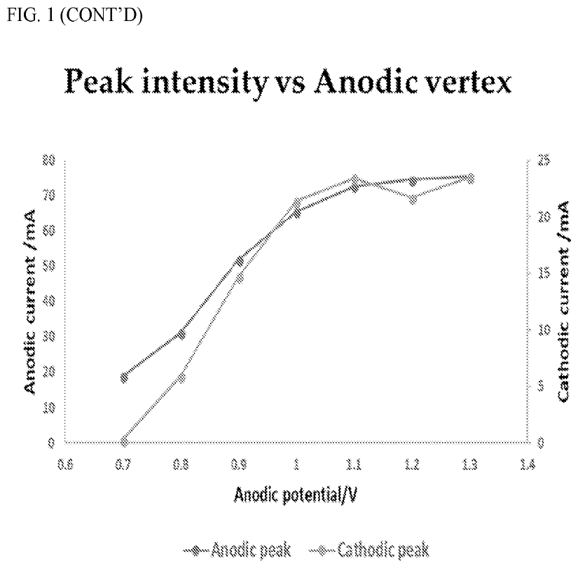

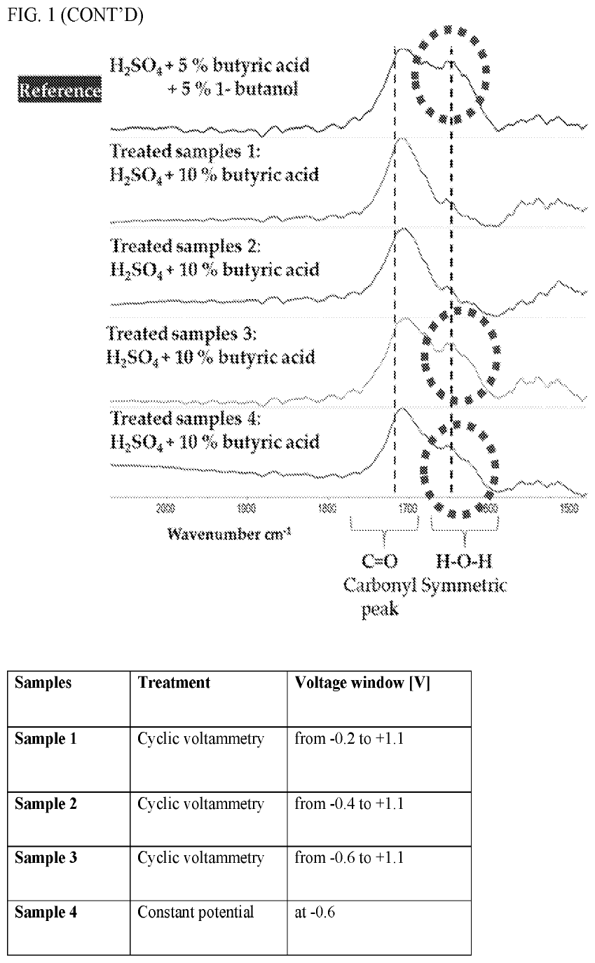

Treated samples were blindly evaluated by a panel of 6 trained volunteers. It was concluded that a cyclic polarization between −0.6 V and 1.1 V vs. EAg/AgCl modulates butyric acid solutions and results in fruitier and more pleasant samples. Fourier-transform infrared (FTIR) spectroscopy was used to characterize the treated samples and indicated the presence of butanol (FIG. 1), which correlates with the sensory analysis. Another important interest for generating butanol from butyric acid is the increase of the detection threshold (0.001 ppm per volume for butyric acid (see, Leonardos, G.; et al., Journal of the Air Pollution Control Association 1969, 19 (2), 91-95) and 0.49 ppm per volume for butanol (see, Albrecht, J.; et al., Chemical Senses 2008, 33 (5), 461-467), which makes butanol less offending.

As shown in FIG. 1, the higher the anodic vertex potential, the higher the cathodic current→Activation of functional groups and/or increase of surface area. As shown in FIG. 1, above 1.1V, the functional groups peak intensity (˜0.1V) ceases to increase→Different functional groups are activated above this potential. As shown in FIG. 1, peak intensity linearly varies with square root of scan rate→Diffusion control process. Stirring was shown to increase the reaction rate. FIG. 1 shows the conditions resulting in successful modulation of butyric acid odorant molecules to butanol. Fourier-transform infrared (FTIR) spectroscopy confirmed the generation of butanol (circled peak) following treatment of butyric acid by cyclic voltammetry. Results indicate that a cathodic vertex lower than −0.4V is required to drive the electrochemical reduction of butyric acid to butanol. FIG. 1 further shows the sample, treatment type (e.g., cyclic voltammetry or constant potential), and voltage window for these experiments.

### Example III

This example describes the electrochemical modulation of para-cresol odorant molecules to 4-hydrobenzaldehyde.

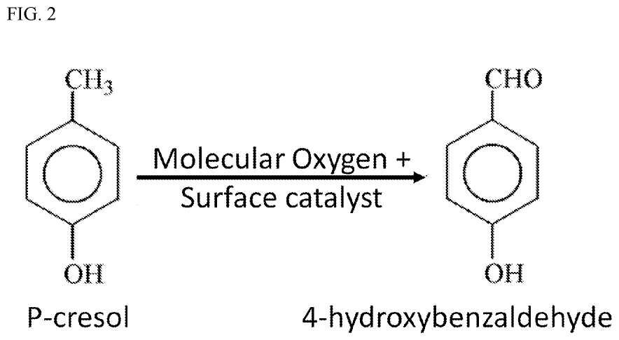

Experiments were conducted to investigate the electrochemical behavior of p-cresol, a malodorant used to reconstitute fecal odor (see, Chappuis Charles, J. F.; et al., Flavour and Fragrance Journal 2015, 31 (1), 95-100). An aim was to generate 4-hydroxybenzaldehyde by electrochemical oxidation (FIG. 2). While p-cresol is a major component in pig odor, its oxidized counterpart is described as a pleasant, woody type odor. Chemical oxidation of p-cresol by molecular oxygen has been reported in literature (see, Wang, F.; et al., Advanced Synthesis & Catalysis 2004, 346 (6), 633-638). It was anticipated that the formation of oxygen at anodic potential coupled with the surface functionalization of graphite electrodes in the same potential range will allow an efficient conversion of p-cresol into 4-hydroxybenzaldehyde.

Various concentrations of p-cresol were contacted with graphite or platinum electrochemically active surface area. Electrochemical modulation of para-cresol odorant molecules was detected on the graphite electrochemically active surface but not the platinum electrochemically active surface thereby indicating the necessity of a carbon based electrochemically active surface area.

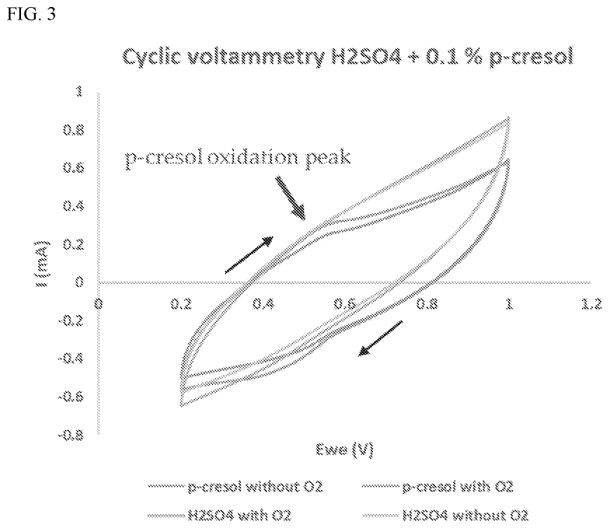

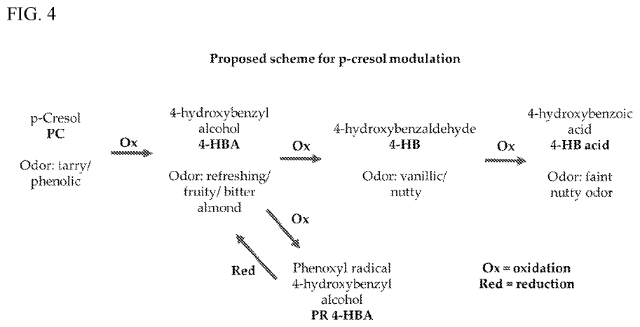

FIG. 3 shows cyclic voltammetry of H₂SO₄+0.1% p-cresol. p-cresol oxidation peak was identified at 0.55 V with cyclic voltammetry for 0.1% p-cresol in 0.5N H₂SO₄. Oxygen saturation was shown to have a catalytic effected as indicated by the increased oxidation peak. FIG. 4 provides a mechanism for p-cresol oxidation.

These experiment demonstrate the successful modulation of para-cresol odorant molecules to a mixture of less offending odorants including 4-hydrobenzaldehyde through electrochemical modulation.

### Example IV

Odorous molecules are highly volatile. Therefore, experiments will be conducted wherein vapor emanating from concentrated mixtures of oxidants will be drained into a customized electrochemical cell equipped with a gas-diffusion electrode (GDE) (FIG. 5). The use of a GDE is of advantage for applications in which gaseous substrates, analytes or products are involved (see, Horst Angelika, E. W.; et al., Biotechnology and Bioengineering 2015, 113 (2), 260-267). A main advantage is to circumvent potential odorants' solubility problem. Due to the fact that GDEs have high specific electroactive areas, theses electrode types are an appropriate starting point for the scale-up. Additional experiments will scale-up the electrochemical setup with an adequate electrode surface-to-volume ratio to treat various mixtures of odorant molecules. In particular, the conversion efficiency of cyclic voltammetry (different scan rates and potential vertices) will be evaluated and compared to static polarization with the aim to minimize energy, reaction byproducts and treatment time.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes.

What is claimed is:

1. A device comprising one or more electrochemically active surface areas, wherein each of the one or more electrochemically active surface areas is configured to electrochemically modulate the smell associated with a specific odorant molecule.

2. The device of claim 1, further comprising an air in-flow portion configured to direct air having odorant molecules to the one or more electrochemically active surface areas.

3. The device of claim 1, further comprising an air out-flow portion configured to direct electrochemically modulated odorant molecules out of the device.

4. The device of claim 1, wherein the device is configured such that it can be programmed to electrochemically modulate the smell associated with specific odorant molecules so as to obtain a desired smell within a setting.

5. The device of claim 1, wherein the device is configured such that it can be directed to electrochemically modulate the smell associated with specific odorant molecules so as to inhibit an undesired smell within a setting.

6. The device of claim 1, wherein one or more of the electrochemically active surface areas is a carbon-based electrochemically active surface area.

7. The device of claim 6, wherein the carbon-based electrochemically active surface area is a graphite-based electrochemically active surface area.

8. The device of claim 1, wherein one or more of the electrochemically active surface areas comprises an electrode.

9. The device of claim 1, wherein one or more of the electrochemically active surface areas is a gas diffusion electrode.

10. The device of claim 1, wherein one or more of the electrochemically active surface areas is any type of electric cell capable of modulating the chemical structure of odorant molecules upon contact with such odorant molecules.

11. The device of claim 1, wherein one or more of the electrochemically active surface areas is a voltaic cell.

12. The device of claim 1, wherein one or more of the electrochemically active surface areas is in contact with an acidic solution.

13. The device of claim 12, wherein one or more of the electrochemically active surface areas is in contact with sulfuric acid.

14. The device of claim 1, wherein one or more of the electrochemically active surface areas is in contact with a basic/alkaline substance.

15. The device of claim 1, the one or more of the electrochemically active surface areas are configured to apply an electric current to contacted odorant molecules which electrochemically modulates one or more functional groups associated with the one or more odorant molecules.

16. The device of claim 15, wherein the one or more of the electrochemically active surface areas are configured to electrochemically modulate ester functional groups to one or more of aldehyde, ketone, alkane, carboxylic acid, and alcohol derivatives.

17. The device of claim 15, wherein the one or more of the electrochemically active surface areas are configured to electrochemically modulate linear terpene functional groups to terpene derivatives.

18. The device of claim 15, wherein the one or more of the electrochemically active surface areas are configured to electrochemically modulate cyclic terpene functional groups to terpene derivatives.

19. The device of claim 15, wherein the one or more of the electrochemically active surface areas are configured to electrochemically modulate aromatic functional groups to aromatic derivatives.

20. The device of claim 15, wherein the one or more of the electrochemically active surface areas are configured to electrochemically modulate amine functional groups amide derivatives.

21. The device of claim 15, wherein the one or more of the electrochemically active surface areas are configured to electrochemically modulate carboxylic acid functional groups to one or more of aldehyde, ketone, alkane, ester, and alcohol derivatives.

22. The device of claim 15, wherein the one or more of the electrochemically active surface areas are configured to electrochemically modulate alcohol functional groups to one or more of aldehyde, carboxylic acid, alkane, ester, and ketone derivatives.

23. The device of claim 15, wherein the one or more of the electrochemically active surface areas are configured to electrochemically modulate aldehyde functional groups to one or more of aldehyde, carboxylic acid, alkane, ester, and ketone derivatives.

24. The device of claim 15, wherein the one or more of the electrochemically active surface areas are configured to electrochemically modulate thiol functional groups to sulfide derivatives.

25. The device of claim 15, wherein the one or more of the electrochemically active surface areas are configured to electrochemically modulate ketone functional groups to one or more of aldehyde, carboxylic acid, alkane, ketone, and ester derivatives.

26. The device of claim 15, wherein the one or more of the electrochemically active surface areas are configured to electrochemically modulate lactone functional groups to one or more of diol derivatives.

27. The device of claim 1, wherein the one or more electrochemically active surface areas are configured to apply a constant electric potential (e.g., at approximately −0.6 V) to contacted odorant molecules.

28. The device of claim 1, wherein the one or more electrochemically active surface areas are configured to apply cyclic voltammetry (e.g., from approximately −0.2 V to +1.1 V) to contacted odorant molecules.

29. The device of claim 15, wherein the one or more electrochemically active surface area modulates the chemical structure of odorant molecules through reducing functional groups on the one or more odorant molecules.

30. The device of claim 15, wherein the one or more electrochemically active surface area modulates the chemical structure of odorant molecules through oxidizing functional groups on the one or more odorant molecules.

31. A method of modulating one or more types of odorant molecules within a setting, comprising providing a device as described in claim 1, directing one or more types of odorant molecules within setting with the one or more electrochemically active surface areas of the device.

32. The method of claim 31, wherein the modulation of the one or more types of odorant molecules results in a modulated smell within the setting.

33. The method of claim 31, wherein one or more of the odorant molecules are selected from geranyl acetate, methyl formate, methyl acetate, methyl proprionate, methyl propanoate, fructone, methyl butyrate, methyl butanoate, ethyl acetate, hexyl acetate, ethyl methylphenylglycidate, ethyl butyrate, ethyl butanoate, isoamyl acetate, pentyl butyrate, pentyl butanoate, pentyl pentanoate, octyl acetate, benzyl acetate, and methyl anthranilate.

34. The method of claim 33, wherein one or more of the one or more electrochemically active surfaces are configured to electrochemically modulate ester functional groups associated with the one or more odorant molecules.

35. The method of claim 31, wherein one or more of the odorant molecules are selected from myrcene, geraniol, nerol, citral, lemonal, geranial, neral, citronellal, citronellol, linalool, and nerolidol.

36. The method of claim 35, wherein one or more of the one or more electrochemically active surfaces are configured to electrochemically modulate linear terpene functional groups associated with the one or more odorant molecules.

37. The method of claim 31, wherein one or more of the odorant molecules are selected from limonene, camphor, menthol, carvone, terpineol, alpha-lonone, thujone, and eucalyptol.

38. The method of claim 37, wherein one or more of the one or more electrochemically active surfaces are configured to electrochemically modulate cyclic terpene functional groups associated with the one or more odorant molecules.

39. The method of claim 31, wherein one or more of the odorant molecules are selected from benzaldehyde, eugenol, cinnamaldehyde, ethyl maltol, vanillin, anisole, anethole, estragole, and thymol.

40. The method of claim 39, wherein one or more of the one or more electrochemically active surfaces are configured to electrochemically modulate aromatic functional groups associated with the one or more odorant molecules.

41. The method of claim 31, wherein one or more of the odorant molecules are selected from trimethylamine, putrescine, diaminobutane, cadaverine, pyridine, indole, and skatole.

42. The method of claim 41, wherein one or more of the one or more electrochemically active surfaces are configured to electrochemically modulate amine functional groups associated with the one or more odorant molecules.

43. The method of claim 31, wherein one or more of the odorant molecules is butyric acid.

44. The method of claim 43, wherein one or more of the one or more electrochemically active surfaces are configured to electrochemically modulate carboxylic acid functional groups associated with the one or more odorant molecules.

45. The method of claim 31, wherein one or more of the odorant molecules are selected from p-cresol, furaneol, 11-hexanol, cis-3-hexen-1-ol, and menthol.

46. The method of claim 45, wherein one or more of the one or more electrochemically active surfaces are configured to electrochemically modulate alcohol functional groups associated with the one or more odorant molecules.

47. The method of claim 31, wherein one or more of the odorant molecules are selected from acetaldehyde, hexanal, cis-3-hexenal, furfural, hexyl cinnamaldehyde, isovaleraldehyde, anisic aldehyde, and cuminaldehyde.

48. The method of claim 47, wherein one or more of the one or more electrochemically active surfaces are configured to electrochemically modulate aldehyde functional groups associated with the one or more odorant molecules.

49. The method of claim 31, wherein one or more of the odorant molecules are selected from thioacetone, allyl thiol, benzyl mercaptan, (methylthio)methanethiol, ethanethiol, ethyl-mercaptan, 2-methyl-2-propanethiol, butane-1-thiol, grapefruit mercaptan, methanethiol, and furan-2-ylmethanethiol.

50. The method of claim 49, wherein one or more of the one or more electrochemically active surfaces are configured to electrochemically modulate thiol functional groups associated with the one or more odorant molecules.

51. The method of claim 31, wherein one or more of the odorant molecules are selected from cyclopentadecanone, dihydrojasmone, 6-acetyl-2,3,4,5-tetrahydropyridine, oct-1-en-3-one, and 2-acetyl-1-pyrroline.

52. The method of claim 51, wherein one or more of the one or more electrochemically active surfaces are configured to electrochemically modulate ketone functional groups associated with the one or more odorant molecules.

53. The method of claim 31, wherein one or more of the odorant molecules are selected from gamma-nonalactone, gamma-decalactone, delta-octalactone, jasmine lactone, massoia lactone, wine lactone, and sotolon.

54. The method of claim 51, wherein one or more of the one or more electrochemically active surfaces are configured to electrochemically modulate lactone functional groups associated with the one or more odorant molecules.

55. The method of claim 31, wherein one or more of the odorant molecules are selected from dimethyl trisulfide, zinc phosphide, methylphosphine, dimethylphosphine, diacetyl, acetoin, nerolin, tetrahydrothiophene, 2,4,6-trichloroanisole, and odorant molecules having a substituted pyrazine functional group.

56. The method of claim 31, wherein the setting is an industrial setting.

57. The method of claim 31, wherein the setting is within a room no larger than 250 square feet.

58. A system or kit comprising one or more of the devices described in claim 1 and instructions for use of the one or more devices.
